(19) United States
(12) Patent Application Publication
(10) Pub. No.: US 2024/0244308 A1
(43) Pub. Date: Jul. 18, 2024

(54) IMAGING SYSTEMS AND METHODS OF USE THEREOF

(71) Applicant: Baracoda, Neuilly-sur-Seine (FR)

(72) Inventors: Thomas Serval, Neuilly-sur-Seine (FR); Olivier Giroud, Neuilly-sur-Seine (FR); Elodie Brient-Litzler, Neuilly-sur-Seine (FR)

(21) Appl. No.: 18/574,206

(22) PCT Filed: Jun. 30, 2022
(86) PCT No.: PCT/IB2022/056086

§ 371 Date: Dec. 26, 2023

Related U.S. Application Data
(60) Provisional application No. 63/216,523, filed on Jun. 30, 2021.

(51) Int. Cl. H04N 23/55; H04N 23/51

(52) U.S. Cl. CPC H04N 23/55; H04N 23/51

(57) ABSTRACT

A system includes a handle, a camera attachment, and a docking station. The camera attachment is configured to be coupled to the handle, and includes an image sensor. The docking station includes a housing that defines a first aperture. The first aperture is configured to receive at least a portion of the camera attachment therein. When the camera attachment is coupled to the handle and not received in the first aperture, the image sensor is configured to generate first image data that is reproducible as an image of a first target area located outside the housing of the docking station. When the camera attachment is received in the first aperture, the image sensor is disposed within the housing of the docking station and is configured to generate second image data that is reproducible as an image of a second target area located within the housing of the docking station.

### CROSS-REFERENCE TO RELATED APPLICATIONS

This application claims priority from and the benefit of U.S. Provisional Patent Application Ser. No. 63/216,523, filed on Jun. 30, 2021, titled “Imaging Systems and Methods of Use Thereof,” which is hereby incorporated by reference herein in its entirety.

### TECHNICAL FIELD

The present disclosure relates generally to imaging systems and methods of use thereof, and more particularly, to imaging systems and methods of use thereof to analyze a target area.

### BACKGROUND

Analyzing a target area such as a region of a user's skin can be beneficial to detect various abnormalities, such as moles, lesions, skin cancers, etc. However, it can be difficult to accurately and consistently analyze the region of the user skin, and to keep the system being used free from contamination. Thus, new systems and methods of use therein are needed.

### SUMMARY

According to some implementations of the present disclosure, a system includes a handle, a camera attachment, and a docking station. The camera attachment is configured to be coupled to the handle, and includes an image sensor. The docking station includes a housing that defines a first aperture. The first aperture is configured to receive at least a portion of the camera attachment therein. When the camera attachment is coupled to the handle and not received in the first aperture, the image sensor is configured to generate first image data that is reproducible as an image of a first target area located outside the housing of the docking station. When the camera attachment is received in the first aperture, the image sensor is disposed within the housing of the docking station and is configured to generate second image data that is reproducible as an image of a second target area located within the housing of the docking station.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

FIG. 1 is a functional block diagram of an imaging system, according to some implementations of the present disclosure;

FIG. 2A is an assembled view of an imaging system, according to some implementations of the present disclosure;

FIG. 2A is an exploded view of the imaging system of FIG. 2A, according to some implementations of the present disclosure;

FIG. 3A is an exploded view of the imaging system of FIG. 2A that further includes a docking station, according to some implementations of the present disclosure;

FIG. 3B is a cross-sectional view of the docking station of FIG. 3A when the components of the imaging system are coupled to the docking station, according to some implementations of the present disclosure;

FIG. 4 is a perspective view of a first alignment mechanism for use with the imaging system of FIG. 2A, according to some implementations of the present disclosure;

FIG. 5A is a perspective view of a second alignment mechanism for use with the imaging system of FIG. 2A when in a first orientation, according to some implementations of the present disclosure;

FIG. 5B is a perspective view of the second alignment mechanism for use with the imaging system of FIG. 2A when in a second orientation, according to some implementations of the present disclosure;

FIG. 6A is a first view of a user using the imaging system of FIG. 2A, according to some implementations of the present disclosure;

FIG. 6B is a second view of a user using the imaging system of FIG. 2A, according to some implementations of the present disclosure; and

FIG. 6C is a third view of a user using the imaging system of FIG. 2A, according to some implementations of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

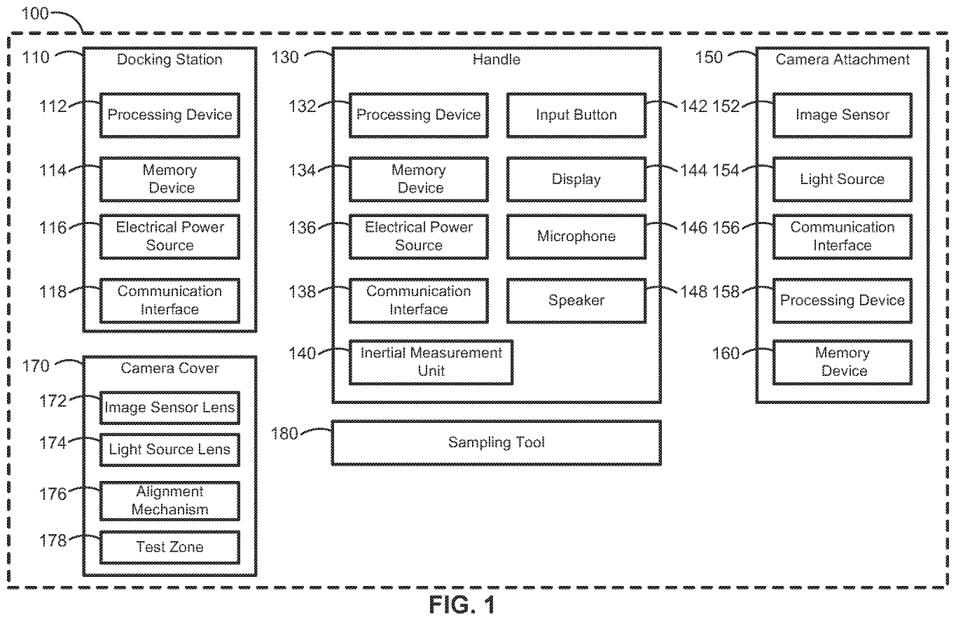

Referring to FIG. 1, a system 100, according to some implementations of the present disclosure is illustrated. The system 100 cam be used to analyze a user's skin and/or hair. System 100 includes a docking station 110, a handle 130, a camera attachment 150, and a camera cover 170. The handle 130, the camera attachment 150, and the camera cover 170 are all configured to be removably coupled to the docking station 110. The docking station 110 can include any combination of a processing device 112, a memory device 114, an electrical power source 116, and a communications interface 118. The processing device 112 can include any number of suitable processing devices, such as a central processing unit (CPU), a microcontroller, etc. The memory device 114 can similarly include any suitable number of memory devices, such as a random or serial access memory device, a hard drive, a solid state drive, a flash memory device, a random-access memory (RAM) device, a read-only memory (ROM) device, etc.

In some implementations, the electrical power source 116 is a rechargeable battery. In other implementations, the electrical power source 116 includes a plug configured to connect to mains power (e.g., via a wall socket) and any other circuitry required to generate a voltage usable by various components of the system 100 (such as transformers, rectifiers, etc.). In some implementations, the electrical power source 116 can include the plug to connect to mains power, the required circuitry, and the rechargeable battery, such that the docking station 110 can still operate when not connected to mains power. The communications interface 118 can include any number of interfaces for connecting the docking station 110 to other components of the system 100, and/or to other components not illustrated in FIG. 1. In some implementations, the communications interface 118 includes a wireless communications interface (such as a WiFi interface, a Bluetooth interface, or both) to allow the docking station 110 to communicate with other components in a wireless fashion. In some implementations, the communications interface 118 can include a wired communications interface (such as a universal serial bus (USB) interface, a universal asynchronous receiver-transmitter (UART) interface, a serial peripheral interface (SPI), an inter-integrated circuit (I²C) interface, or any combination thereof) to allow the docking station 110 to communication with other components in a wired fashion. In further implementations, the communications interface 118 includes both a wireless communications interface and a wired communications interface.

The handle 130 can include a processing device 132, a memory device 134, an electrical power source 136, a communications interface 138, an inertial measurement unit (IMU) 140, one or more input buttons 142, a display 144, a microphone 146, and a speaker 148. The processing device 132 is similar to the processing device 112, and can include any number of suitable processing devices. The memory device 134 is similar to the memory device 114, and can include any number of suitable memory devices. The electrical power source 136 of the handle 130 will generally include only a rechargeable battery, but in some implementations, could additionally or alternatively include a plug configured to be connected to mains power and associated circuitry. The communications interface 138 is similar to the communications interface 118, and can include any number or combination of wireless communication interfaces and wired communication interfaces. In some implementations, the communications interface 138 can be used to electrically connect the electrical power source 136 (e.g., the rechargeable battery) to another device, either to transmit electrical power to that other device (e.g., to charge the device) or to receive electrical power from that other device (e.g., to be charged by the device). For example, the communications interface 138 can be used to connect the handle 130 to the docking station 110, so that the docking station 110 can charge the rechargeable battery in the handle 130.

The IMU 140 is used to generate data that is representative of the location of the handle 130, the movement of the handle 130, forces experienced by the handle 130, etc. In some implementations, the IMU 140 is a six-axis IMU, which includes a tri-axis accelerometer and a tri-axis gyroscope, a tri-axis accelerometer and a tri-axis magnetometer, or a tri-axis gyroscope and a tri-axis magnetometer. In other implementations, the IMU 140 is a nine-axis IMU, which includes a tri-axis accelerometer, a tri-axis gyroscope, and a tri-axis magnetometer.

The one or more input buttons 142 can include any number or combination of buttons. The input buttons 142 can include push buttons, sliding switches, capacitive touch buttons, or any other types of buttons. The input buttons 142 can be momentary buttons (e.g., the button returns to its initial state after the applied force is removed), maintained buttons (e.g., the button remains in its new state after the applied force is removed), or a combination thereof. The one or more input buttons 142 can be used to receive input from the user, and cause the processing device 132 to perform various functions. The display 144 can include any number or combination of displays, including a liquid crystal display (LCD) screen and/or an organic light emitting diode (OLED) screen. The display 144 can also include a light emitting diode (LED) or an array of LEDs. The display 144 can be used to communicate various information to the user.

The microphone 146 can generate acoustic data that is reproducible as one or more sound(s) that occur during the use of the system 100. The acoustic data can be used, for example, to detect the sound of running water in the area around the system 100, such as from a sink, a bathtub, and/or a shower. The speaker 148 outputs sound waves that are audible to the user. The speaker 148 can be used to communicate information to the user.

The camera attachment 150 can be coupled to the handle 130. In some implementations, the camera attachment 150 can be removable coupled to the handle 130, such that the camera attachment 150 can be repeatedly attached to and detached from the handle 130. In other implementations, the camera attachment 150 is configured to be permanently affixed to the handle 130. The camera attachment 150 can include any combination of an image sensor 152, a light source 154, and a communications interface 156. The image sensor 152 can include any suitable number or combination of image sensors, such as a red-green-blue (RGB) image sensor, a multispectral image sensor, and/or a hyperspectral image sensor. As discussed in more details herein, the image sensor 152 is configured to generate image data that is reproducible as an image of a target area. The target area could be the user's skin, the user's hair, or some other object. The light source 154 is configured to emit light to aid in illuminating the target area. The light source 154 can include any number and combination of light sources, such as white light emitting diodes (LEDs), blue LEDs, near-ultraviolet (near-UV) LEDs, and/or infrared (IR) LEDs.

The communications interface 156 is similar to the communications interface 118 and 138, and can include any number or combination of wireless communication interfaces and wired communication interfaces. In some implementations, the communications interface 156 can be used to electrically connect an electrical power source (such as the electrical power source 136 of the handle 130) to the camera attachment 150, which is then used to power the image sensor 152 and the light source 154. The camera attachment 150 may also include a processing device 158 and a memory device 160 that can be powered by an electrical power source (such as the electrical power source 136 of the handle 130).

The camera cover 170 is configured to fit over the camera attachment 150. The camera cover 170 can include an image sensor lens 172 that is aligned with the image sensor 152 of the camera attachment 150, when the camera cover 170 is attached to the camera attachment 150. The image sensor lens 172 can serve to focus and/or magnify the image sensor 152. The camera cover 170 may also include a light source lens 174 that aligns with the light source 154 of the camera attachment 150. The light source lens 174 can focus/direct the light from the light source 154, or can simply allow the light from the light source 154 to pass therethrough. Finally, the camera cover 170 can include an alignment mechanism 176 that is configured to aid in aligning the image sensor 152 with respect to the target area and/or aid in positioning the image sensor 152 a desired distance away from the target area, as is discussed in more detail herein.

The camera cover 170 can further include one or more test zones 178. Each of the test zones 178 is configured to aid in characterizing one or more properties of the target area, which may be a portion of the user's skin, a portion of the user's hair, etc. In some implementations, characterizing a property of the target area includes determining the value of the property of the target area. For example, a property of the target area can be a pH level (e.g., the pH level of the user's skin), and characterizing this property can include determining the actual pH level of the target area. The test zones 178 can thus act as sensors that determine (and/or aid in determining) the value of the property of the target area. In other implementations, the property is the presence or absence of a particular material, and characterizing the property includes determining whether the material is present, and/or determining how much of the material is present. In further implementations, characterizing one or more properties of the target area includes determining the chemical/biochemical composition of the target area. For example, the test zones 178 can be used to determine the composition of a user's skin and/or hair.

In some implementations, the test zones 178 include some type of reactive material (such as a chemical reagent, a biological reagent, a biochemical reagent, a nano-material, etc.) that is configured to react to the presence of a predetermined material located at and/or near the target area, to react to a property of the target area having a specific value, or both. In some of these implementations, the exposure of the test zones 178 to the target area can cause the optical properties of the test zones 178 to change. The optical properties can include the color (sometimes referred to as chromaticity), hue, colorfulness, reflectivity, transmissivity, fluorescence, scattering angle, frequency of reflective light, wavelength of reflective light, etc. For example, the test zones 178 can be colorimetric sensors that react to the presence of some material at or near the target area (e.g., the test zones 178 can change colors based on the presence of different materials), and/or react to some property of the target area (e.g., the test zones 178 can change to a specific color based on the pH level of the target area). In another example, the test zones 178 can be become fluorescent in response to the presence of some material at or near the target area (such as a PCR (polymerase chain reaction) test that utilizes fluorescence to measure the amount of a substance). In this example, the system 100 can utilize a light source (such as the light source 154 of the camera attachment 150) to cause the test zones 178 to fluoresce, which can then be measured. In further implementations, the reactive material is configured to cause one or more non-optical properties of the test zones 178 to change in response to being exposed to the target area.

In some implementations, the test zones 178 do not contain any type of reactive material, but are still configured to react to the presence of some material at or near the target area, and/or the target area having some predetermined value of a property. In these implementations, the test zones 178 could be configured to undergo changes in various optical properties, and/or to experience changes in other types of properties as well. In some implementations, the test zones 178 are one or more portions of the surface of the camera cover 170 that have had some type of reactive material placed thereon. In other implementations, the test zones 178 are one or more portions of the surface of the camera cover 170 onto which a separate sensor or device has been placed.

The system 100 can further include a sampling tool 180. The sampling tool 180 includes a collection area, and is configured to collect physical material from a sampling area, such that some of the physical material is disposed at, within, and/or near the collection area of the sampling tool 180. In some cases, the sampling area from which the sampling tool 180 collects the physical material is the same target area that the image sensor 152 generates image data of. For example, the target area and the sampling area could both be the same portion of the user's skin and/or hair, and the collected physical material can be biological material from the user (e.g., a skin sample, a hair sample, etc.). The collected physical material can be analyzed to aid in characterizing some sort of property of the sampling area (e.g., the value of a property, the presence and/or amount of physical material present at the sampling area, etc.). The sampling tool 180 can have any suitable size and/or shape. For example, the sampling tool 180 may be formed as a scraper that is used to scrape physical material from the sampling area. The sampling tool 180 could also be formed as a hook, a claw, etc.

System 100 can be used to analyze skin and/or hair of the user, for example by aiming the camera attachment 150 at a target area of the user that includes a portion of the user's skin that the user wishes to analyze, and/or a portion of the user's hair that the user wishes to analyze. As is discussed in more detail herein, the image sensor 152 of the camera attachment 150 can be used to generate image data reproducible as an image of the portion of the user's skin and/or hair. The image data can then be processed to analyze the portion of the user's skin and/or hair and determine a variety of metrics related thereto. System 100 can also be used to analyze the image sensor lens 172 of the camera cover 170, when both the camera attachment 150 and the camera cover 170 are coupled to the docking station 110.

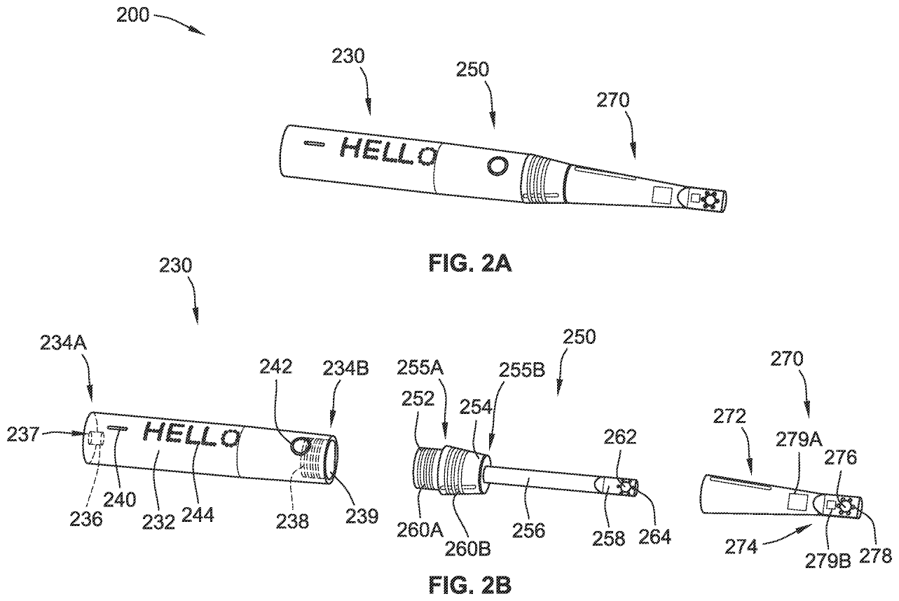

FIG. 2A shows a system 200 assembled for use, and FIG. 2B shows an exploded view of system 200. System 200 is a specific implementation of system 100 from FIG. 1. As shown, system 200 includes a handle 230, a camera attachment 250, and a camera cover 270. The handle 230 is the same as or similar to the handle 130 of system 100. The camera attachment 250 is the same as or similar to the camera attachment 150 of system 100. The camera cover 270 is the same as or similar to the camera cover 170 of system 100.

The handle 230 includes a housing 232 having a first end 234A and a second end 234B. An electrical contact 236 is formed at the first end 234A, and a plurality of electrical contacts 238 are formed at the second end 234B. The electrical contact 236 and the plurality of electrical contacts 238 can form all or part of a communication interface of the handle 230 (similar to communications interface 138 of handle 130). The electrical contact 236 is located within a depression 237 that is formed at the first end 234A of the housing 232. The depression 237 can have a generally cylindrical shape, such that the depression 237 has a circular cross-section. The plurality of electrical contacts 238 are located within a depression 239 that is formed at the second end 234B of the housing 232. The depression 239 can have a generally cylindrical shape, such that the depression 239 has a circular cross-section. In the illustrated implementation, the plurality of electrical contacts 238 are formed as a series of annular rings. In this manner, the plurality of electrical contacts 238 are formed on the portion of the housing 232 that forms the outer annular wall of the depression 239. The electrical contact 236 could similarly be an annular electrical contact, but could have other shapes as well, such as a single point contact.

The plurality of electrical contacts 238 can have any number of electrical contacts, including two electrical contacts, three electrical contacts, four electrical contacts, five electrical contacts, six electrical contacts, or more. Further, while FIG. 2B shows a plurality of electrical contacts 238 formed at the second end 234B of the handle 230, in some implementations the handle 230 instead has a single electrical contact 238 formed at the second end 234B.

The handle 230 further includes an indicator light 240, an input button 242, and a display 244. The indicator light 240 can include any suitable light source (such as an LED), and can be used to indicate specific information to a user of the system 200. For example, in some implementations, the indicator light 240 can be activate (e.g., emitting light) when the handle 230 is electrically connected to another component (such as via electrical contact 236 or the plurality of electrical contacts 238), and can be inactive (e.g., not emitting light) when the handle 230 is not electrically connected to another component. In other implementations, the indicator light 240 could instead change to different colors to indicate the connection status of the handle 230. In still further implementations, the indicator light 240 could be activated and inactivated according to a pre-defined pattern to indicate the desired information to the user. For example, in some implementations, the indicator light 240 could be solid (e.g., constantly activated) when the handle 230 is connected to another component, but then could blink according to a pattern when data is being transferred between the handle 230 and the other component.

The input button 242 can be similar to the one or more input buttons 142 of system 100, and can be a physical button, a physical switch, a capacitive touch button, or another type of button. The input button 242 can be used to receive input from the user. For example, in some implementations, the user can press the input button 242 to activate and deactivate the system 200. The display 244 can be similar to the display 144 of the handle 130. In the illustrated implementation, the display 244 is formed as a series of LEDs arranged to spell out the word “HELLO.” The display 244 can be activated when the handle 230 is activated (e.g., turned on by the user), to indicate to the user that the handle 230 has been activated. However, the display 244 could also include a screen, such as an LCD screen or an OLED screen, similar to display 144.

The handle 230 may include a variety of other components disposed within the housing 232 that are not shown in FIGS. 2A and 2B. For example, the handle 230 can include an electrical power source (which could be a rechargeable battery, and is generally similar to electrical power source 136), one or more processing devices (which could be similar to processing device 132), one or more memory devices (which could be similar to memory device 134), additional wired and/or wireless communication interfaces (such as a WiFi interface or a Bluetooth interface), circuitry required to power the various components of the handle 230 (or other components of system 200), and other devices or components.

The camera attachment 250 is similar to the camera attachment 150 of system 100, and can be used to generate image data that is reproducible as an image of a target area, which may be some area of a user's body, and/or some other area of the system 200. The camera attachment 250 includes a base region 252, a shoulder region 254, a neck region 256, and a head region 258. The base region 252 has a generally cylindrical shape with a circular cross-section, and is sized to be received within the depression 239 formed at the second end 234B of the housing 232 of the handle 230, to thereby removably couple the camera attachment 250 to the handle 230. The shoulder region 254 is generally frustum-shaped with a circular cross-section. The end 255A of the shoulder region 254 nearest to the base region 252 has a larger diameter than the diameter of the depression 239 of the handle 230. Thus, when the base region 252 is inserted into the depression 239, the end 255A of the shoulder region 254 can aid in indicating to the user when the base region 252 is fully inserted. In some implementations, the end 255A of the shoulder region 254 can also aid in preventing the base region 252 from being inserted further than necessary into the depression 239. The diameter of the shoulder region 254 narrows between the end 255A and the opposing end 255B.

The neck region 256 forms an elongated rod that separates the head region 258 from the shoulder region 254. In the illustrated implementation, the neck region 256 has a generally cylindrical shape with a constant diameter. However, the neck region 256 can have other shapes in other implementations. In the illustrated implementation, the head region 258 has a half-cylindrical shape that is flat on one side, and curved on the other side. The cross-section of the head region 258 is a half-circle. However, in other implementations, the head region 258 can have different shapes. The camera attachment 250 includes an image sensor 262 to generate image data, and a ring of light sources 264 disposed at the head region 258 that can aid in illuminating the target area. In the illustrated implementation, the image sensor 262 is located within the center of the ring of light sources 264. However, other arrangements can be used in other implementations. For example, a single light source 264 can be located next to the image sensor 262, multiple image sensors 262 can be used, etc. The image sensor 262 can be the same as or similar to image sensor 152 of system 100. The light sources 264 can be the same as or similar to the light source 154 of system 100. The light sources 264 can all be the same type of light sources (e.g., all white LEDs), or can be a combination of different light sources 264 that can be used for different applications (e.g., at least one white LED, at least one near-UV LED, and at least one IR LED). The camera attachment 250 may include one or more processing devices that can control the operation of the image sensor 262 and the light sources 264, and/or one or more memory devices that can store image data generated by the image sensor 262.

The camera attachment 250 further includes a plurality of electrical contacts 260A formed on the exterior surface of the base region 252, and a plurality of electrical contacts 260B formed on the exterior surface of the shoulder region 254. When the base region 252A is received within the depression 239 and the camera attachment 250 is coupled to the handle 230, the plurality of electrical contacts 260A mate with the plurality of electrical contacts 238 of the handle 230, to thereby electrically connect the handle 230 and the camera attachment 250. The electrical contacts 260A can thus form part of a communications interface of the camera attachment 250 (similar to the communications interface 156 of the camera attachment 150 of system 100). Generally, the plurality of electrical contacts 238 can be electrically connected to various components of the handle 230, including the electrical power source of the handle 230. Similarly, the plurality of electrical contacts 260A can be electrically connected to the image sensor 262 and the light sources 264.

Thus, when the camera attachment 250 is coupled to the handle 230 and the electrical contacts 260A mate with the electrical contacts 238, the electronic components of the camera attachment 250 are electrically coupled to the electronic components of the handle 230. This allows the electrical power source of the handle 230 to provide electrical power to the image sensor 262 and the light sources 264. This can also allow any processing devices of the handle 230 to control the operation of the image sensor 262 and the light sources 264, and/or for the image data generated by the image sensor 262 to be stored in any memory devices of the handle 230, in addition to or as an alternative to any control and/or storage performed by any processing devices and memory devices in the camera attachment 250.

The plurality of electrical contacts 260A will generally match the plurality of electrical contacts 238. Thus, each of the electrical contacts 260A has a generally annular shape, and there is a single corresponding electrical contact 260A for each single one of the electrical contacts 238. Thus, the electrical contacts 260A can include one electrical contact, two electrical contacts, three electrical contacts, four electrical contacts, five electrical contacts, six electrical contacts, or more.

The electrical contacts 238 and 260A can be used to electrically connect the handle 230 and the camera attachment 250 in a variety of ways. In some implementations, the plurality of electrical contacts 238 and the plurality of electrical contacts 260A each include six electrical contacts. One electrical contact from each group can be used as a power pin, one electrical contact from each group can be used as a ground pin, and the remaining four electrical contacts can be used as data pins. These four data pins can be implemented as a variety of different data communication interfaces, such as USB Type A, USB Type B, UART, SPI, I²C, and other communication interfaces.

The camera attachment 250 can include a second plurality of electrical contacts 260B formed on the exterior surface of the shoulder region 254. The plurality of electrical contacts 260B can have an annular shape similar to the electrical contacts 260A, and can also have the same number of electrical contacts as the plurality of electrical contacts 260A. The electrical contacts 260B can also be electrically connected to the image sensor 262, the light sources 264, and any other electronic components in the camera attachment 250, such as processing devices and/or memory devices. However, the electrical contacts 260B do not mate with the electrical contacts 238 when the camera attachment 250 is coupled to the handle 230. Instead, as discussed further herein, the electrical contacts 260B can be used to electrically connect the camera attachment 250 to other components of the system 200.

The camera cover 270 is formed from a neck region 272 and a head region 274. The neck region 272 has a frustum shape similar to the shoulder region 254 of the camera attachment 250. The head region 274 has a half-cylindrical shape similar to the head region 258 of the camera attachment 250. The camera cover 270 is configured to fit over the camera attachment 250 when the system 200 is assembled for use by a user. Thus, the neck region 272 and the head region 274 of the camera cover are both generally hollow, and are sized to receive therein at least a portion of the neck region 256 and the head region 258 of the camera attachment 250.

As can be seen by comparing FIGS. 2A and 2B, when the system 200 is assembled for use, an end 273 of the neck region 272 will abut the end 255B of the shoulder region 254. The camera attachment 250 and the camera cover 270 can include corresponding mating features that removably couple the camera cover 270 to the camera attachment 250. In this manner, the camera cover 270 completely covers the neck region 256 and the head region 258 of the camera attachment 250, such that the neck region 256 and the head region 258 are shielded in all directions by the camera cover 270. However, in other implementations, the camera cover 270 can be designed such the end 273 of the camera cover 270 does not reach the end 255B of the shoulder region 254 when the camera attachment 250 is received within the camera cover 270. In further implementations, the camera cover 270 can be designed such that the end 273 does reach the end 255B of the shoulder region 254, but the camera cover 270 includes various openings or apertures, such that the neck region 256 and the head region 258 are open to the exterior through these openings or apertures.

The camera cover 270 further includes an image sensor lens 276 and a plurality of light source lenses 278. The image sensor lens 276 can be the same as or similar to the image sensor lens 172 of system 100. The light source lenses 278 can each be similar to or the same as the light source lens 174 of system 100. When the camera cover 270 is removably coupled to the camera attachment 250, the image sensor lens 276 of will be aligned with the image sensor 262, and each of the plurality of light source lenses 278 will be aligned with one of the light sources 264. The image sensor lens 276 can be used to focus, magnify, or otherwise alter the image of the target area that can be produced from the image data generated by the image sensor 262. The light source lenses 278 can also be used to focus the light emitted by the light sources 264 onto a smaller portion of the target area. However, in other implementations, the light source lenses 278 can be used to diffuse the light emitted by the light sources 264, so as to illuminate a larger portion of the target area. In further implementations, light source lenses 278 can be made from a generally transparent material, such that the light emitted by the light sources 264 is not altered when passing through the light source lenses 278. In even further implementations, the camera cover 270 can include apertures instead of the light source lenses 278, to allow the light emitted by the light sources 264 to pass through the camera cover 270 undisturbed.

The camera cover 270 includes two test zones 279A and 279B. Each of the test zones 279A and 279B can be the same as or similar to the test zone 178 of the system 100 in FIG. 1. Test zone 279A is located on the surface of the neck region 272 of the camera cover 270. Test zone 279B is located on the surface of the head region 274 of the camera cover 270. Each of the test zones 279A and 279B can be used to characterize a property of a target area as described above with respect to the test zone 178. For example, if a user grasps the handle 230 such that the image sensor 262 of the camera attachment 250 is near the target area, the test zones 279A and 279B can be used to analyze the target area, such as by determining the value of some property (e.g., a pH level), detecting the presence and/or the amount of some material at or near the target area (e.g., bacteria), etc. The test zones 279A and 279B can be colorimetric sensors that change color to indicate the value of the property and/or the presence of a predetermined material.

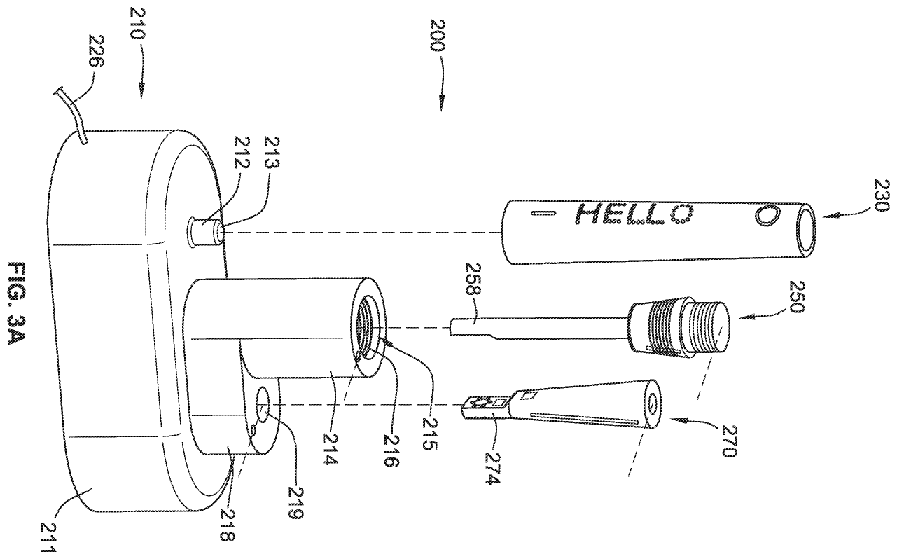

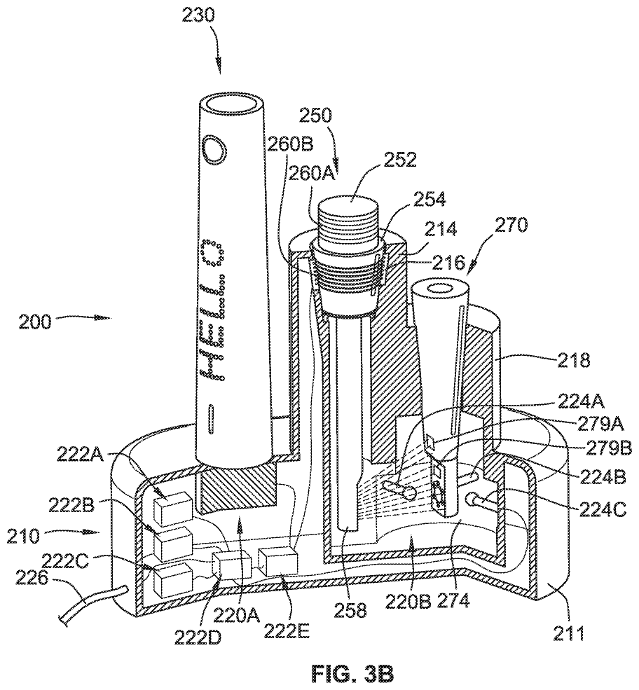

Referring now to FIGS. 3A and 3B, the system 200 can further include a docking station 210, which can be similar to or the same as docking station 110 of system 100. The docking station 210 includes a housing 211 that defines a first attachment point 212, a second attachment point 214, and a third attachment point 218. The first attachment point 212, the second attachment point 214, and the third attachment point 218 can be used to couple the handle 230, the camera attachment 250, and the camera cover 270, respectively, to the docking station. The docking station 210 also includes an electrical plug 226 that can be connected to mains power (e.g., a wall outlet).

The first attachment point 212 is formed as a protrusion that includes an electrical contact 213 disposed on the upper surface of the protrusion. When the handle 230 is coupled to the docking station 210 via the first attachment point 212, the first attachment point 212 is received in the depression 237 (FIG. 2B) formed at the first end 234A of the housing 232 of the handle 230. The electrical contact 236 of the handle 230 is configured to mate with the electrical contact 213 formed at the top of the first attachment point 212, to thereby electrically connect the various components of the handle 230 to the docking station 210.

The second attachment point 214 defines an aperture 215 that is configured to receive a portion of the camera attachment 250 therein, to thereby couple the camera attachment 250 to the docking station 210. Similarly, the third attachment point 218 defines an aperture 219 that is configured to receive a portion of the camera cover 270 therein, to thereby couple the camera cover 270 to the docking station 210. The second attachment point 214 includes a plurality of electrical contacts 216 on the interior portion of the second attachment point 214 that forms the periphery of the aperture 215. As shown in FIG. 3B, when the camera attachment 250 is inserted into the aperture 215, the electrical contacts 216 of the second attachment point 214 will mate with the electrical contacts 260B that are formed on the shoulder region 254 of the camera attachment 250. The camera attachment 250 can thus be electrically connected to the docking station 210 by inserting the camera attachment 250 into the aperture 215 of the second attachment point 214. Thus, the electrical contacts 260B of the camera attachment 250 can form part of the communications interface of the camera attachment 250. Similarly, the electrical contacts 216 of the second attachment point 214 can form part of a communications interface of the docking station 210 (which can be the same as or similar to the communications interface 118 of the docking station 110 of system 100).

The electrical contacts 216 and 260B can be used to electrically connect the docking station 210 and the camera attachment 250 in a variety of ways. In some implementations, the plurality of electrical contacts 216 and the plurality of electrical contacts 260B each include six electrical contacts. One electrical contact from each group can be used as a power pin, one electrical contact from each group can be used as a ground pin, and the remaining four electrical contacts can be used as data pins. These four data pins can be implemented as a variety of different data communication interfaces, such as USB Type A, USB Type B, UART, SPI, I²C, and other communication interfaces.

Similar to the second attachment point 214, attachment point 218 defines an aperture 219 that is configured to receive a portion of the camera cover 270 therein, to thereby couple the camera cover 270 to the docking station 210. However, the second attachment point 214 generally does not include any electrical contacts.

FIG. 3B shows a cross-sectional view of the docking station 210 when the handle 230, the camera attachment 250, and the camera cover 270 are all coupled to the docking station 210. As shown, both the head region 258 of the camera attachment 250, and the head region 274 of the camera cover 270 are disposed within the hollow interior of the housing 211. The docking station 210 also includes a number of components 222A, 222B, 222C, 222D, and 222E that can be disposed within the hollow interior of the housing 211. In the illustrated implementation, the interior of the housing 211 is divided into two different internal compartments 220A and 220B. The components 222A-222E are disposed in compartment 220A, while the head region 258 of the camera attachment 250 and the head region 274 of the camera cover 270 are disposed in compartment 220B.

In some implementations, component 222A is a processing device, which can be the same as or similar to processing device 112 of the docking station 110. In some implementations, component 222B is a memory device, which can be the same as or similar to memory device 114 of the docking station 110. In some implementations, component 222C is a rechargeable battery, which can be the same as or similar to the rechargeable battery that can form part of the electrical power source 116 of the docking station 110. In some implementations, component 222D is a WiFi interface, which can be the same as or similar to the WiFi interface that can form part of communications interface 118 of the docking station 110. In some implementations, component 222E is a Bluetooth interface, which can be the same as or similar to the Bluetooth interface that can form part of communications interface 118 of the docking station 110.

As shown in FIGS. 3A and 3B, the various components 222A-222E of the docking station 210 can be electrically connected to the handle 230 via the electrical contact 213 of the docking station 210, and the electrical contact 236 of the handle 230. The components 222A-222E can also be electrically connected to the camera attachment 250 via the electrical contacts 216 and the electrical contacts 260B. Thus, the components 222A-222E of the docking station 210 (and any other components that the docking station 210 may include) can be electrically connected to various components of the handle 230 and the camera attachment 250. In some implementations, the rechargeable battery 222C and/or the electrical plug 226 of the docking station 210 can be used to charge the rechargeable battery of the handle 230, and or provide power to components of the handle 230, such as the indicator light 240, the input button 242, the display 244, and/or any processing devices and memory devices in the handle 230. In some implementations, the rechargeable battery 222C and/or the electrical plug 226 of the docking station 210 can be used to provide power to the image sensor 262, the light sources 264, and/or any processing devices and memory devices in the camera attachment 250.

In some implementations, the processing device 222A and the memory device 222B can be used to control other components of the system 200 and store generated data. For example, processing device 222A could control the image sensor 262 and the light sources 264 when the camera attachment 250 is coupled to the docking station. Thus, even though not connected to the handle 230, the image sensor 262 can generate image data that is reproducible as an image of a target area when the camera attachment 250 is coupled to the docking station. In another example, the image sensor 262 can generate image data (of the original target area and/or a different target area) when the camera attachment 250 is coupled to the handle 230 and the handle 230 is coupled to the docking station 210. In a further example, memory device 222B can be used to store any image data generated by the camera attachment 250, whether the image data was (i) previously generated and stored in a memory device of the handle 230, (ii) previously generated and stored in a memory device of the camera attachment 250, or (iii) generated in real-time as the camera attachment 250 is coupled to the docking station 210.

As shown in FIG. 3B, when the camera attachment 250 and the camera cover 270 are coupled to the docking station 210, the image sensor 262 and the light sources 264 can be aimed at the neck region 272 and/or the head region 274 of the camera cover 270 (e.g., so that the neck region 272 and/or the head region 274 of the camera cover 270 is the target area for the camera attachment 250). The docking station 210 can include alignment mechanisms 224A, 224B, and 224C that aid in ensuring that the image sensor lens 276 disposed on the head region 274 of the camera cover 270 is facing toward the image sensor 262. The image sensor 262 (which could be powered and controlled by the components of the docking station 210) can thus be used to generate image data that is reproducible as an image of the neck region 272 and/or the head region 274 of the camera cover 270. As discussed in more detail herein, the image data can be used to analyze the neck region 272 and/or the head region 274 of the camera cover 270, for example for the presence of bacteria or other unwanted material.

Thus, the camera attachment 250 can be used to generate image data of a first target area outside of the housing 211 of the docking station 210 (e.g., a target area on the user's body), and can also be used to generate image data of a second target area inside of the housing 211 of the docking station 210 (e.g., the neck region 272 and/or the head region 274 of the camera cover 270). As is further shown in FIG. 3B, the image sensor 282 and the light sources 264 can also be aimed at the test zones 279A and 279B of the camera cover 279. Thus, in addition to the test zones 279A and 279B being used to directly characterize some property of some target area outside of the housing 211 of the docking station 210, the image sensor 262 can be used to generate image data that is reproducible as an image of the test zones 279A and 279B. The test zones 279A and 279B themselves can be analyzed to characterize one or more properties of a first target area (e.g., a target area on the user's body), and image data associated with the second target area (the test zones 279A and 279B when the camera cover 270 is disposed within the housing 211 of the docking station 210) can be analyzed to characterizing one or more properties of the first target area. In some implementations, the light sources 264 can be used to illuminate the test zones 279A and 279B after they have been exposed to the target area(s), and the image sensor 262 can generate image data of the test zones 279A and 279B to measure the resulting reaction. For example, the test zones 279A and 279B may be configured to have or develop fluorescent properties after being exposed to the target area. The light sources 264 can illuminate the test zones 279A and 279B to cause them to fluoresce, which can then be measured by the image sensor 262.

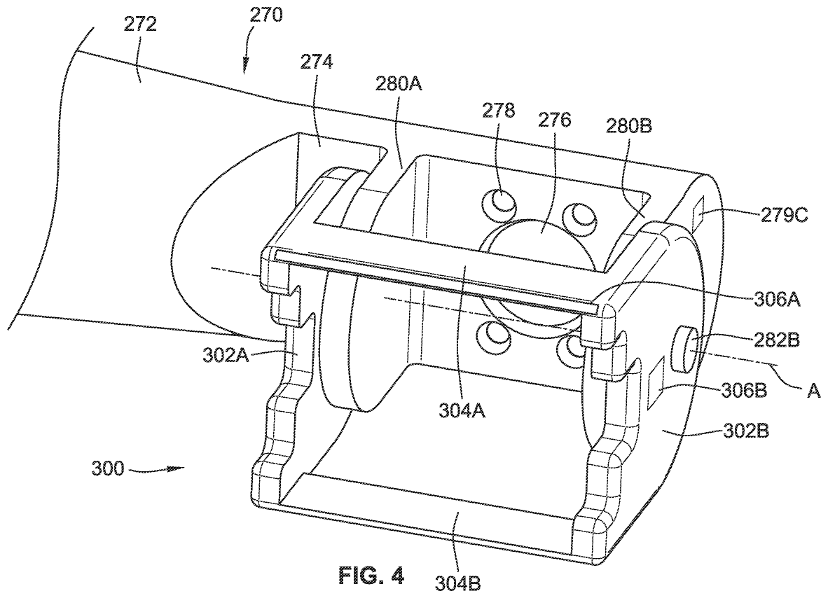

Referring now to FIG. 4, when the system 200 is used to analyze the user's skin, the system 200 can include an alignment mechanism that aids the user in positioning the image sensor 262 of the camera attachment 250. FIG. 4 illustrates an alignment mechanism 300 that can be coupled to the camera cover 270. Alignment mechanism 300 can be the same as or similar to the alignment mechanism 176 in FIG. 1. In the illustrated implementations, the head region 274 of the camera cover 270 includes a pair of protrusions 280A and 280B located on either side of the image sensor lens 276 and the light source lenses 278. The alignment mechanism 300 includes sidewalls 302A and 302B that are connected by crossmembers 304A and 304B. Sidewall 302A is coupled to the protrusion 280A, while sidewall 302B is coupled to the protrusion 280B.

Together, the proximal surfaces of the sidewall 302A, the sidewall 302B, the crossmember 304A, and the crossmember 304B form a generally flat surface which can contact the portion of the user's skin that the user wishes to analyze (e.g., the target area). The alignment mechanism 300 aids in ensuring that the image sensor 262 and the light source 264 are pointing toward the target area on the user's skin, and also in ensuring that the user does not place the image sensor 262 too close to the target area on the user's skin. In the illustrated implementation, alignment mechanism 300 can also rotate about the head region 274 of the camera cover 270, to allow the image sensor 262 and the light source 264 to analyze the target area of the user's skin from different angles. As shown, the protrusion 280B can include a boss 282B extending therefrom that can be inserted into an aperture defined by the sidewall 302B. The protrusion 280A can include a similar boss 282A extending therefrom that can be inserted into an aperture defined by the sidewall 302A, which is visible in FIGS. SA and SB. The alignment mechanism 300, and more specifically the sidewalls 302A and 302B, can rotate about the bosses that extend from the protrusions 280A and 280B. As shown, the alignment mechanism 300 can rotate about an axis A that runs through the center of the bosses 282A and 282B.

As is shown in FIG. 4, the alignment mechanism 300 can include one or more test zones that can be used to characterize a property of a target area. The alignment mechanism 300 includes a test zone 306A located on the crossmember 304A, and a test zone 306B located on the sidewall 302B. FIG. 4 also shows an additional test zone 279C located at the distal end of the head region 274 of the camera cover 270. Each of these test zones can be the same as or similar to the test zone 178 of the system 100 in FIG. 1 and/or the test zones 279A and 279B of the camera cover 270 and can be used in the same or similar fashions.

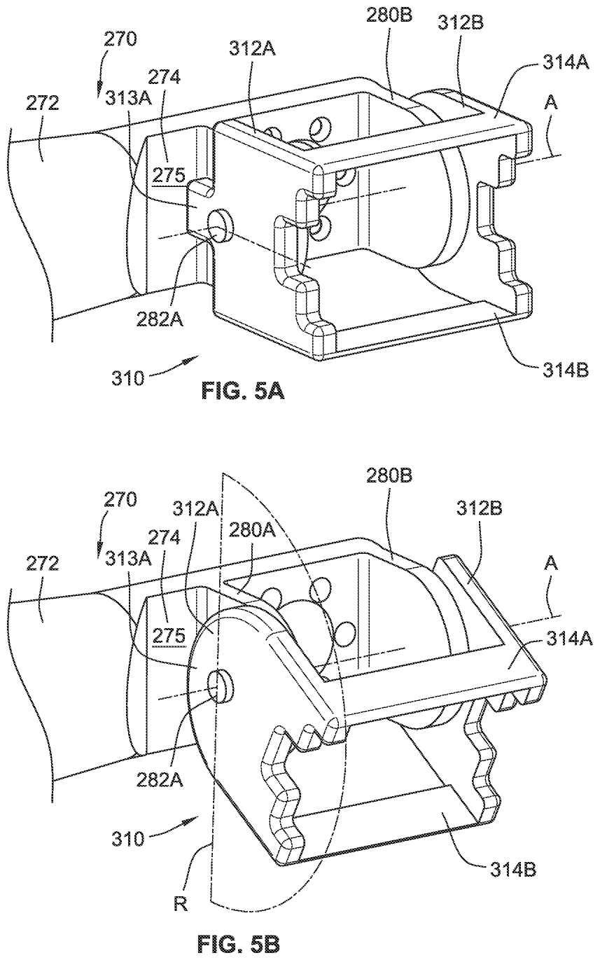

FIGS. 5A and 5B show an alignment mechanism 310 that is similar to alignment mechanism 300. However, alignment mechanism 310 has sidewalls with two different shapes, which allows the alignment mechanism 310 to be coupled to the head region 274 of the camera cover 270 in two different orientations. In the first orientation illustrated in FIG. 5A, the alignment mechanism 310 cannot rotate relative to the head region 274 of the camera cover 270, whereas in the second orientation illustrated in FIG. 5B, the alignment mechanism 310 can rotate relative to the head region 274 of the camera cover 270.

The alignment mechanism 310 includes two sidewalls 312A and 312B that are coupled to the protrusions 280A and 280B of the camera cover 270, and two cross-members 413A and 314B that connect the sidewalls 312A and 312B. Similar to the embodiment in FIG. 4, the head region 274 of the camera cover 270 includes protrusion 280A and 280B that include respective bosses 282A and 282B extending therefrom. The sidewalls 312A and 312B can be coupled to protrusions 280A and 280B via the bosses 282A and 282B, similar to alignment mechanism 300. However, sidewalls 312A and 312B have two different shapes. Sidewall 312A has a generally flat terminus 313A, while sidewall 312B has a generally curved terminus 313B.

When the alignment mechanism 310 is in the first orientation illustrated in FIG. 5A, the sidewall 312B is coupled to protrusion 280B, and is thus disposed past the distal end of the camera cover 270 that is formed by the protrusion 280B. As such, the curved terminus 313B of the sidewall 312B does not abut or contact any portion of the head region 274. However, because the sidewall 312A is coupled to the protrusion 280A, the flat terminus 313A of the sidewall 312A generally abuts a corresponding flat surface 275 of the head region 274. If the user attempted to rotate the alignment mechanism 310 relative to the head region 274 of the camera cover 270, the flat terminus 313A of the sidewall 312A would contact the flat surface 275, and prevent this rotation from occurring.

However, when the alignment mechanism 310 is moved to the second orientation illustrated in FIG. 5B, the coupling between the sidewalls 312A, 312B and the protrusions 280A, 280B is swapped. Thus, the flat terminus 313A of sidewall 312A is disposed past the distal end of the camera cover 270 that is formed by the protrusion 280B, and the curved terminus 313B of sidewall 312B abuts the flat surface 275 of the head region 274. When the user attempts to rotate the alignment mechanism 310 relative to the head region 274 of the camera cover 270, the curved terminus 313B of the sidewall 312B is able to continue rotating past the flat surface 275 without contact the flat surface 275. Thus, the alignment mechanism 310 is able to rotate about the axis A that runs through the center of the bosses 282A and 282B. FIG. 5B shows the range R through which the alignment mechanism 310 is able to rotate.

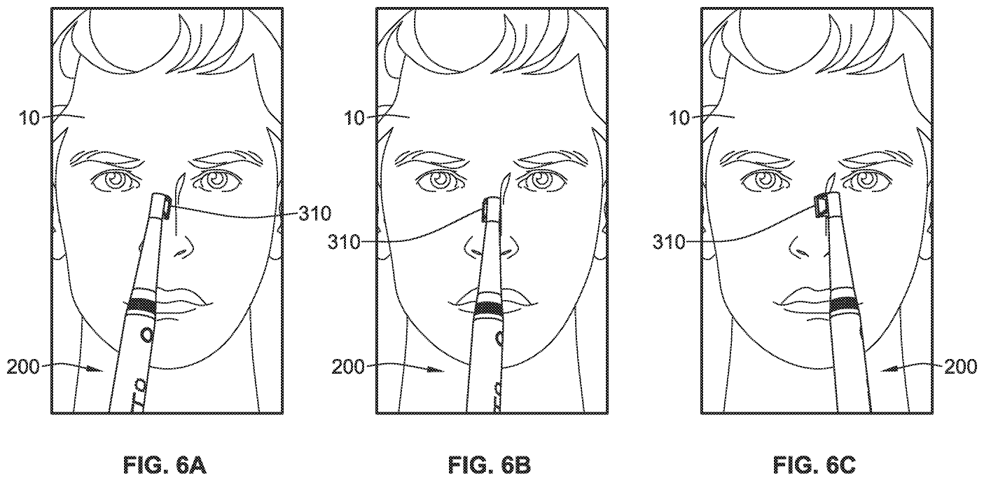

FIGS. 6A, 6B, and 6C show different stages of a user 10 using the system 200 to analyze a target area of the user's skin. In FIGS. 6A, 6B, and 6C, the alignment mechanism used with system 200 can rotate, and is thus either alignment mechanism 300, or alignment mechanism 310 when in the orientation illustrated in FIG. 5B. In FIG. 6A, the user 10 is holding the system 200 up to the bridge of their nose, such that the alignment mechanism 300/310 contacts the bridge of their nose. This ensures that the image sensor 262 of the camera attachment 250 is positioned an appropriate distance away from the user's skin, so that the image data generated by the image sensor 262 can create accurate images of the user's skin and allow for accurate analysis. In FIG. 6A, the system 200 is positioned relative to alignment mechanism 300/310 such that the image sensor 262 is positioned over the right side of the bridge of the user's nose. In FIG. 6B, the user 10 has rotated the system 200 relative to the alignment mechanism 300/310. In this position, the image sensor 262 is still positioned the correct distance away from the user's skin, due to the alignment mechanism alignment mechanism 300/310. However, the image sensor 262 is now generally positioned over the center of the bridge of the user 10's nose. In FIG. 6C, the user 10 has further rotated the system 200 relative to the alignment mechanism 300/310. In this position, the image sensor 262 is again positioned the correct distance away from the user's skin, due to the alignment mechanism 300/310. However, the image sensor 262 is now generally positioned over the left side of the bridge of the user 10's nose.

Thus, the alignment mechanisms 300 and 310 can allow the user to position the image sensor 262 of the system 200 an appropriate distance away from the target area of their skin, such that the generated image data can be used to accurately analyze the target area. However, the alignment mechanism 300 and 310 can also allow the user to gradually move the image sensor 262 to nearby target areas, without requiring the user to re-position the system 200 and the image sensor 262. The alignment mechanisms 300 and 310 can thus aid in generating image data of a continuous region on the user's skin spanning multiple target areas.

Generally, system 200 can be used in a variety of different ways to analyze the user's skin and/or hair. In some implementations, the image data generated by the image sensor 262 of the camera attachment 250 can be stored in the memory device of the handle 230, if the image data is generated while the camera attachment 250 is coupled to the handle 230, and neither component is coupled to the docking station 210. In response to the handle 230 being coupled to the docking station 210, the image data can be transferred from the memory device of the handle 230 to the memory device of the docking station 210. The processing device of the docking station 210 can then perform more advanced analysis on the image data. In other implementations, the generated image data remains in a memory device of the camera attachment 250, and then is transferred to the memory device of the docking station 210 in response to the camera attachment 250 being coupled to the docking station 210.

In some implementations, when the camera attachment 250 and the camera cover 270 are coupled to the docking station 210 such that the image sensor 262, the light sources 264, the image sensor lens 276, and the light source lenses 278 are disposed within the housing 211 of the docking station 210, the processing device of the docking station 210 can control the image sensor 262 and the light sources 264. The docking station 210 can cause the light sources 264 to illuminate the target area, which includes the image sensor lens 276 and the light source lens 278. The docking station 210 can cause the image sensor 262 to generate image data of the image sensor lens 276 and the light source lenses 278. This image data can be stored in the memory device of the docking station 210.

In still other implementations, the camera attachment 250 can remain coupled to the handle 230 when the handle 230 is coupled to the docking station 210. In these implementations, the docking station 210 can power components of both the handle 230 and the camera attachment 250, including recharging any rechargeable batteries in the handle 230 and the camera attachment 250. The processing device of the docking station 210 can then control the image sensor 262 and the light sources 264, for example to generate image data of another target area. The user could thus use the system 200 without actually having to hold onto the handle 230 and the camera attachment 250. Any image data generate in this configuration can be stored in any combination of memory devices of the camera attachment 250, the handle 230, and the docking station 210.

In some implementations, the docking station 210 can be configured to receive therein a physical object used by the user, such as a hair brush, a comb, a lipstick tube, a mascara applicator, a makeup brush, or other device. When the physical object is disposed within the interior of the housing 211 of the docking station 210, the camera attachment 250 can be inserted into the docking station 210 and generate image data of the physical object. In some implementations, the physical object can be received within the same aperture of the housing 211 that receives the camera cover 270. In these implementations, an end of the physical object will be disposed adjacent to the image sensor 262 of the camera attachment 250, and thus serve as a target area for the image sensor 262. In other implementations, the housing 211 of the docking station 210 can include an additional aperture to receive the physical object, so long as at least a portion of the physical object will be within view of the image sensor 262 of the camera attachment 250.

In some implementations, the physical object includes a sampling tool, which may be the same as or similar to the sampling tool 180 of the system 100 in FIG. 1. The sampling tool is used to collect physical material from a sampling area. In some cases, the sampling area is or includes the same target area that the image sensor 262 generates image data of. For example, if the target area of the image sensor 262 is a portion of the user's skin, the sampling area could include that same portion of the user's skin. In some implementations, the sampling area of the sampling tool includes multiple areas. For example, the sampling area can include a first area outside of the housing 211 of the docking station 210 and a second area inside of the housing 211 of the docking station 210. In some of these implementations, the first sampling area is the same first target area on the user's body that the image sensor 262 generates image data of, and the second sampling area is the same second target area on the camera cover 270 within the housing 211 that the image sensor 262 generates image data of. Thus, the sampling tool can be used to collect physical material from a portion of the user's body (such as their skin or hair), and also from the camera cover 270 that was in close proximity to that same portion of the user's body when image data 262 of that portion of the user's body was being generated.

The collection area of the sampling tool (where the collected physical material resides) can be disposed within the housing 211 of the docking station 210 in view of the image sensor 262 of the camera attachment 250. Thus, similar to the test zones 279A-279C of the camera cover 270, the collection area of the sampling tool can collect physical material from a first target area outside of the housing 211 of the docking station 210, and then act as a second target area within the docking station 210. The image data generated by the image sensor 262 can be analyzed to provide additional information related to the first target area.

In some implementations, components such as the docking station 210, the handle 230, the camera attachment 250, and the camera cover 270 can be formed from a durable, waterproof material. A nano self-cleaning coating can be applied to these components, which provides hydrophilic, photocatalytic, and anti-static properties.

The image data can be processed in a variety of ways to analyze the target area. For example, when analyzing skin, the image data can be processed to identify anomalies such as UV spots, beauty spots, moles, lesions, skin cancers, etc. Any identified anomalies can be measured to determine the size, diameter, color, contrast on the border, shape, etc. Anomalies can be tracked over time to measure any changes, and images can be shared with a healthcare provider. The image data can also be processed to identify cuts, scrapes, bruises, scars, etc. The image data can also be processed to identify and measure wrinkles and pores, and track wrinkles and pores over time. A detail map of the user's facial features can also be generated. When analyzing hair, the image data can be processed to measure the thickness of the user's hair (e.g., the thickness of individual strands or groups of strands), and/or the dryness of the user's hair. The image data can also be processed to identify the presence of bacteria or other unwanted organisms on the camera cover. The docking station can include a sanitization unit configured to aid in sanitizing the camera cover. The sanitization unit could include a UV light, sanitizing liquid, etc.

In some implementations, various components of the disclosed systems can be used with different components. For example, the handle 230 and the docking station 210 could be used with a different attachment other than the camera attachment 250.

One or more elements or aspects or steps, or any portion(s) thereof, from one or more of any of claims 1-70 below can be combined with one or more elements or aspects or steps, or any portion(s) thereof, from one or more of any of the other claims 1-70 or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1. A system comprising:
a handle;
a camera attachment configured to be coupled to the handle, the camera attachment including an image sensor; and
a docking station including a housing, the housing defining a first aperture configured to receive at least a portion of the camera attachment therein;
wherein when the camera attachment is coupled to the handle and not received in the first aperture, the image sensor is configured to generate first image data that is reproducible as an image of a first target area located outside the housing of the docking station, and
wherein when the camera attachment is received in the first aperture, the image sensor is disposed within the housing of the docking station and is configured to generate second image data that is reproducible as an image of a second target area located within the housing of the docking station.

2. (canceled)

3. The system of claim 1, wherein the image sensor includes a red-green-blue (RGB) image sensor, a multispectral image sensor, a hyperspectral image sensor, or any combination thereof, and wherein the light source includes one or more white light emitting diodes (LEDs), one or more blue LEDs, one or more near-ultraviolet (near-UV) LEDs, one or more infrared (IR) LEDs, or any combination thereof.

4-5. (canceled)

6. The system of claim 3, wherein the handle includes a housing and an electrical power source disposed within the housing, and wherein when the camera attachment is coupled to the handle, the electrical power source of the handle provides electrical power to the image sensor and the light source.

7-9. (canceled)

10. The system of claim 1, wherein the docking station includes an electrical power source, a processing device, and a memory device, each disposed within the housing of the docking station.

11. The system of claim 10, wherein in response to the camera attachment being received within the first aperture of the docking station, the camera attachment is electrically connected to the electrical power source of the docking station, the processing device of the docking station, and the memory device of the docking station, the processing device of the docking station being configured to control the image sensor when the camera attachment is received in the first aperture of the docking station to generate the second image data, the memory device of the docking station being configured to store the second image data.

12-13. (canceled)

14. The system of claim 11, wherein the camera attachment includes a light source, and wherein the processing device of the docking station is configured to control the light source when the camera attachment is received in the first aperture of the docking station to illuminate the second target area.

15. (canceled)

16. The system of claim 10, wherein the docking station further includes a protrusion configured to couple to least a portion of the handle, and wherein the electrical power source of the docking station is configured to charge an electrical power source of the handle when the handle is coupled to the protrusion of the docking station.

17-18. (canceled)

19. The system of claim 16, wherein when the camera attachment is coupled to the handle and the handle is coupled to the protrusion, the image sensor is electrically connected to the electrical power source of the docking station, the processing device of the docking station, and the memory device of the docking station.

20. The system of claim 19, wherein the handle includes a memory device configured to store the first image data generated by the camera attachment when the camera attachment is coupled to the handle and not received by the first aperture of the docking station, and wherein the processing device of the docking station is configured to cause the first image data to be transferred from the memory device of the handle to the memory device of the docking station, when the handle is coupled to the protrusion of the docking station.

21-24. (canceled)

25. The system of claim 1, wherein the camera attachment includes a first set of one or more electrical contacts and a second set of one or more electrical contacts, and wherein:
the first set of one or more electrical contacts of the camera attachment are configured to mate with a corresponding set of one or more electrical contacts of the handle when the camera attachment is coupled to the handle, to thereby electrically connect the camera attachment and the handle; and
the second set of one or more electrical contacts of the camera attachment are configured to mate with a corresponding set of one or more electrical contacts of the docking station when camera attachment is received in the first aperture of the docking station, to thereby electrically connect the camera attachment and the docking station.

26-27. (canceled)

28. The system of claim 1, further comprising a camera cover configured to be removably coupled to the camera attachment, the camera cover including an image sensor lens, wherein in response to the camera cover being coupled to the camera attachment, the image sensor of the camera attachment is aligned with the image sensor lens of the camera cover, such that the image sensor lens is positioned between the image sensor and the first target area.

29. (canceled)

30. The system of claim 28, wherein the docking station includes a second aperture configured to receive at least a portion of the camera cover therein, the image sensor lens of the camera cover being disposed within the housing of the docking station when the camera cover is received in the second aperture of the docking station, wherein the second target area includes the image sensor lens of the camera cover, and wherein the second image data generated by the image sensor of the camera attachment is reproducible as an image of the image sensor lens of the camera cover.

31-33. (canceled)

34. The system of claim 28, wherein the camera cover includes one or more test zones, each of the one or more test zones being configured to aid in characterizing one or more properties of the first target area, and wherein the first target area includes a portion of a user.

35-44. (canceled)

45. The system of claim 34, wherein the second target area includes at least one of the one or more test zones, and wherein the second image data generated by the image sensor of the camera attachment can be analyzed to aid in characterizing the one or more properties of the first target area.

46-53. (canceled)

54. The system of claim 1, wherein the docking station includes a second aperture configured to receive a physical object that is separate from the camera attachment, a portion of the physical object being disposed within the housing of the docking station when the physical object is received in the second aperture of the docking station, and wherein the second target area includes the portion of the physical object disposed within the housing of the docking station, and wherein the second image data generated by the image sensor of the camera attachment is reproducible as an image of the portion of the physical object disposed within the housing of the docking station.

55-56. (canceled)

57. The system of claim 1, wherein the first target area is a portion of skin of a user, a portion of hair of the user, or both, and wherein the first image data is reproducible as an image of the portion of the skin of the user, an image of the portion of the hair of the user, or both.

58-60. (canceled)

61. The system of claim 1, wherein the docking station includes a memory device and a processing device, the memory device being configured to store the first image data, the second image data, or both the first image data and the second image data, the processing device being configured to analyze the first image data, the second image data, or both the first image data and the second image data.

62-63. (canceled)

64. The system of claim 1, further comprising an alignment mechanism coupled to the camera attachment, the alignment mechanism being configured to aid in positioning the image sensor a desired distance away from the first target area.

65. (canceled)

66. The system of claim 64, wherein the alignment mechanism is rotatably coupled to the camera attachment, to thereby allow the camera attachment to be rotated relative to the target area.

67. (canceled)

68. The system of claim 64, wherein the alignment mechanism is configured to be coupled to the camera attachment in a first orientation and a second orientation, the alignment mechanism in the first orientation being rotatable relative to the camera attachment, the alignment mechanism in the second orientation being fixed relative to the camera attachment.

69-70. (canceled)
